(19) United States
(12) Patent Application Publication
(10) Pub. No.: US 2024/0424282 A1
(43) Pub. Date: Dec. 26, 2024

(54) Minimally Invasive Single Port Pulsatile Ventricular Assist Device

(71) Applicant: University of Kentucky Research Foundation, Lexington, KY (US)

(72) Inventors: Dongfang Wang, Lexington, KY (US); Joseph B. Zwischenberger, Lexington, KY (US); Cherry Ballard-Croft, Lexington, KY (US); Li Li, Lexington, KY (US); Jinsong Chen, Lexington, KY (US)

(21) Appl. No.: 18/744,158

(22) Filed: Jun. 14, 2024

Related U.S. Application Data
(60) Provisional application No. 63/521,085, filed on Jun. 14, 2023.

(51) Int. Cl. A61M 60/178; A61M 60/117; A61M 60/268; A61M 60/435; A61M 60/538; A61M 60/554; A61M 60/861; A61M 60/896

(52) U.S. Cl. CPC A61M 60/178; A61M 60/268; A61M 60/435; A61M 60/861; A61M 60/896; A61M 60/117; A61M 60/538; A61M 60/554

(57) ABSTRACT

A ventricular assist device includes a cannula including a lumen. An inlet valve on the cannula is in communication with the lumen. An outlet valve on the cannula is in communication with the lumen. A pump is connected to the lumen and adapted to draw blood through the inlet valve from the ventricle into the lumen and deliver blood from the lumen through the outlet valve into an aorta or pulmonary artery of the a patient into which the device is implanted. The ventricular assist device also includes an anchor including (a) a graft adapted to receive and hold the cannula and (b) an anchor retainer carried on a first end of the graft and adapted to secure the anchor to the ventricle wall.

### RELATED APPLICATIONS

This document claims priority to U.S. Provisional Patent Application Ser. No. 63/521,085, filed on Jun. 14, 2023, which is hereby incorporated by reference.

### GOVERNMENT INTEREST

The invention was made with Government support under grant number 1R43HL158430-01/3200004483 awarded by the National Institutes of Health SBIR. The government has certain rights in the invention.

### TECHNICAL FIELD

This document relates generally to a compact, single port, pulsatile ventricular assist device for ventricle support that employs a minimally invasive single cannulation technique for implantation.

### BACKGROUND

Cardiogenic shock (CS) is a serious condition of reduced cardiac output (CO) with end organ hypoperfusion. Even with recent advances in treatment, CS mortality is still as high as 40-50%. In severe CS, two critical pathophysiological mechanisms lead to patient death: (1) low CO causes end organ hypoperfusion, resulting in multiple organ failure and (2) significantly elevated left ventricle (LV) preload increases LV wall stress, exacerbating myocardial injury and preventing recovery.

Mechanical circulatory support (MCS) is required to restore end organ perfusion and unload the LV, bridging the CS patient to recovery, further treatment, long-term LV assist device (LVAD), or heart transplantation. Peripheral venoarterial extracorporeal membrane oxygenation (VA ECMO) is most often used for severe CS and is the fastest way to reestablish circulation. However, VA ECMO fails to unload the LV in more than 50% of severe CS patients, requiring additional measures to unload the LV. Percutaneous MCS devices have been widely used in severe CS, but their limited circulatory support capacity is often not enough to completely stabilize the circulation of these patients. Non-percutaneous MCS devices supply up to total cardiac support but require an invasive open chest surgery to create connections to the patient's heart and aorta.

Prior art devices of the type described above include the regular diaphragm displacement pump illustrated in FIG. 1. That pump P is connected through (a) an inlet valve IV and inlet cannula IC to the left ventricle of the heart for drainage and (b) the outlet valve OV and outlet cannula OC to the aorta for infusion. During operation, the pump alternates between blood withdrawal and infusion and inlet cannula IC and Outlet cannula OC usage is 50%. In use, the device requires, not one but two separate cannulations and a relatively large pump equipped with both inlet and outlet valves that must be monitored and operated in proper coordination to drain blood from the ventricle and infuse blood in the aorta.

Another prior art device of the type described above is disclosed in, for example, U.S. Pat. Nos. 9,669,144 and 11,648,390, both to Spanier et al. The blood pump disclosed in these patents is not a percutaneous device since it requires a relatively difficult implantation through an anastomosed graft on an axillary artery. As such, the device is contraindicated in patients with severe peripheral vascular disease (atherosclerosis), and significant aortic valve stenosis and in those with mechanical valves. Further, use of the device requires the patient to stay in the intensive care unit (ICU), at great expense, with limited ambulation due to potential for pump dislodgement and continuous purge system requirement.

This document describes a new and improved ventricular assist device characterized by a number of significant advantages over prior art MCS devices. As will be described in greater detail below, these advantages include:
1) Minimally invasive installation: the new and improved ventricular assist device is designed to be implanted through a small thoracotomy and transapical to aorta cannulation or from right ventricular wall into right ventricle to pulmonary artery.
2) Provides total LV support: the new and improved ventricular assist device is able to achieve over 6 L/min mean pumping blood flow to ensure complete re-establishment of collapsed circulation.
3) Enables easy ambulation: the new and improved ventricular assist device includes a very small paracorporeal single port diaphragm displacement pump (spDDP) connected to the heart through only one small transcutaneous VSLC, facilitating very convenient ambulation and allowing patient discharge from ICU and even hospital for at home temporary MCS.

### SUMMARY

In accordance with the purposes and benefits set forth herein, a new and improved ventricular assist device comprises, consists of or consists essentially of: (a) a cannula including a lumen and a first portion adapted to engage a wall of a left or right ventricle of a heart into which the ventricular device is inserted, (b) an inlet valve on the cannula and in communication with the lumen, (c) an outlet valve on the cannula and in communication with the lumen, (d) a pump connected to the lumen of the cannula and adapted to draw blood through the inlet valve from the left or right ventricle into the lumen and deliver blood from the lumen through the outlet valve into an aorta or pulmonary artery of the patient, and (e) an anchor including a graft adapted to receive and hold the cannula and an anchor retainer carried on a first end of the graft and adapted to secure the anchor to a wall of the ventricle.

The ventricular assist device further includes a second portion adapted to cross an aorta valve or pulmonary valve between the left or right ventricle and the aorta or pulmonary artery of the heart into which the ventricular assist device is inserted. The ventricular assist device may further include a cannula retainer carried on the cannula. The anchor may further include at least one tie fastening the graft to the cannula at the cannula retainer.

In at least some of the many possible embodiments, the cannula retainer includes a first retainer ring and a second retainer ring carried on the cannula. The first retainer ring may be positioned inside the ventricle wall and the second retainer ring may be positioned outside the ventricle wall when the device is properly implanted in the heart of the patient. In such embodiments, the anchor may further include at least one tie fastening the graft to the cannula between the second retainer ring and the third retainer ring, or beyond second retainer ring to prevent cannula dislodgement (pulling out).

The ventricular assist device may further include stitching connecting the anchor retainer to the ventricle wall. In at least some embodiments, the graft is adapted to extend from the anchor retainer connected to the ventricle wall through a chest wall of a patient into which the ventricular assist device is implanted.

The ventricular assist device may further include stitching securing the graft to the chest wall at an intermediate point between the first end and a second opposite end of the graft.

In at least some of the many possible embodiments, the anchor retainer is a sewing mat that is stitched to the ventricle wall. In at least some of the many possible embodiments, the graft is a tube made from a medical grade synthetic material appropriate for the intended purpose. When the ventricular assist device is properly implanted for use, the cannula is received and held in the tube by at least one tie fastening the graft to the cannula between the second retainer ring and the third retainer ring, or beyond second retainer ring to prevent cannula dislodgement (pulling out).

More specifically describing the ventricular assist device, the inlet valve is a one-way valve, the outlet valve is a one-way valve and the lumen is a single lumen. The pump is a valveless, single port diaphragm displacement pump. The ventricular assist device may further include a pump drive connected to the pump. That pump drive may be mounted on a cart with wheels for purposes of assisting patient ambulation.

In accordance with yet another aspect, a new and improve anchor is provided. That anchor comprises, consists of or consists essentially of a graft adapted to receive and hold a heart cannula and an anchor on a first end of the graft and adapted to secure the anchor to a ventricle wall of a patient by sewing to ventricular wall.

The anchor may further include at least one tie fastening the graft to the cannula. The anchor may further include stitching connecting the anchor to the ventricle wall. In at least some embodiments, the graft is adapted to extend from the anchor retainer connected to the ventricle wall through a chest wall of the patient. In such embodiments, the anchor may further include stitching securing the graft to the chest wall at an intermediate point between the first end and a second opposite end of the graft.

In at least some of the many possible embodiments, the anchor retainer is a sewing mat that is stitched to the ventricle wall. In at least some of the many possible embodiments, the graft is a tube made from a medical grade synthetic material. In at least some embodiments, the heart cannula is received and held in the tube.

In at least some embodiments, the device may further include a third retainer ring carried on the cannula. In such a device, a securing tie may connect the graft to the cannula between the second retainer ring and the third retainer ring.

In accordance with yet another aspect, a method of providing mechanical circulatory support to a patient, comprises, consists of or consists essentially of the steps of: (a) inserting a cannula of a ventricular assist device through a ventricle wall of the patient, (b) positioning an inlet valve, carried on the cannula, in the ventricle and an outlet valve, carried on the cannula, in an aorta or a pulmonary artery of the patient, (c) supporting the cannula with an anchor, and (d) establishing blood flow through the cannula with a pump adapted to (1) draw blood from the ventricle through the inlet valve and (2) discharge blood through the outlet valve into the aorta or the pulmonary artery.

The method may further include inserting the cannula in a graft of the anchor. The method may further include connecting the cannula to the graft. The method may further include connecting the graft to the ventricle wall and a chest wall of the patient. The method may further include securing an anchor retainer at a first end of the anchor to the ventricle wall. The method may further include connecting the graft to the chest wall between the first end and a second, opposite end of the graft.

The method may further include connecting the pump to a proximal end of the cannula.

The method may further include connecting a pump drive to the pump. The method may further include positioning the pump drive on a cart supported on wheels. Such an arrangement assists with patient ambulance which may play a significant role in patient recovery.

The method may further include making a left thoracotomy through which the cannula is inserted through the left ventricle wall. The method may further include positioning the inlet valve in the left ventricle and the outlet valve in the thoracic aorta.

In other embodiments and for other purposes, the method may further include making a right thoracotomy through which the cannula is inserted through the right ventricle wall. This may be followed by positioning the inlet valve in the right ventricle and the outlet valve in the pulmonary artery.

In the following description, there are shown and described several different embodiments of the new and improved (a) ventricular assist device, (b) anchor for that ventricular assist device and (c) method of providing mechanical circulatory support to a patient using that ventricular assist device. As it should be realized, the ventricular assist device, anchor and method are capable of other, different embodiments and their several details are capable of modification in various, obvious aspects all without departing from the ventricular assist device, anchor and method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate certain aspects of the new and improved (a) ventricular assist device, (b) anchor and (c) method of providing mechanical circulatory support to a patient using that ventricular assist device and together with the description serve to explain certain principles thereof. A person of ordinary skill in the art will readily recognize from the following discussion that alternative embodiments of the insulating materials may be employed without departing from the principles described below.

FIG. 1 is a schematic illustration of a prior art mechanical circulatory support device having two cannulas.

FIG. 2 is a detailed side elevational view of the cannula of the ventricular assist device.

FIG. 3A is a detailed schematic illustration of an inlet valve on the cannula shown in a closed position.

FIG. 3B is a detailed view similar to FIG. 3A but showing the inlet valve in an open position.

FIGS. 4A and 4B are detailed views of an alternative embodiment of the inlet valve shown, respectively, in closed and open positions.

FIG. 5A is a detailed perspective view of an outlet valve at the first or distal end of the cannula wherein the outlet valve is shown in a closed position.

FIG. 5B is a view similar to FIG. 5A but showing the outlet valve in an open position.

FIG. 6 is a schematic block diagram illustrating the drive system for the pump that is attached to the second or proximal end of the cannula.

FIG. 7 is a detailed perspective view of an apical anchor used to receive and hold the cannula in position within a heart of the patient into which the cannula is implanted.

FIGS. 8A and 8B are schematic perspective views illustrating a two different embodiments of the ventricular assist device with the cannula properly implanted within the heart of the patient with the apical anchor receiving and holding the cannula in position with the inlet valve of the cannula in the left ventricle and the outlet valve of the cannula in the aorta.

FIGS. 9A-9C illustrate the pressure waveforms that guide the proper placement of the cannula of the ventricular assist device in the heart of the patient. More specifically, FIG. 9A shows the waveform generated when the catheter introducer is in the left ventricle. FIG. 9B shows the waveform generated when the catheter introducer is in the ascending aorta. FIG. 9C shows the waveform generated when the catheter introducer is misplaced in the left atrium.

FIG. 10 is a schematic illustration of a patient with the ventricular assist device implanted ambulating while pulling the pump drive on a wheeled cart.

Reference will now be made in detail to the present preferred embodiments of the (a) ventricular assist device, (b) anchor and (c) method of providing mechanical circulatory support to a patient using that ventricular assist device.

### DETAILED DESCRIPTION

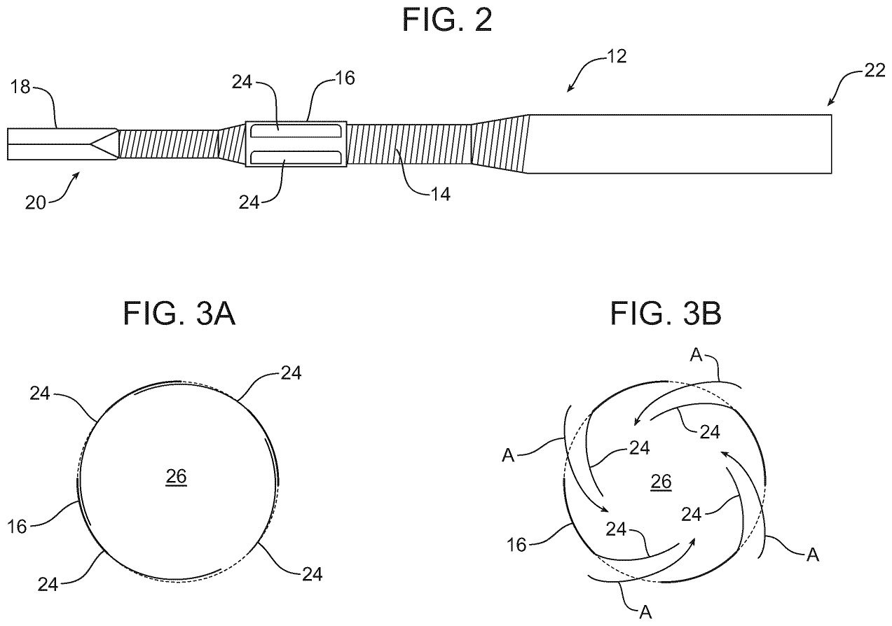

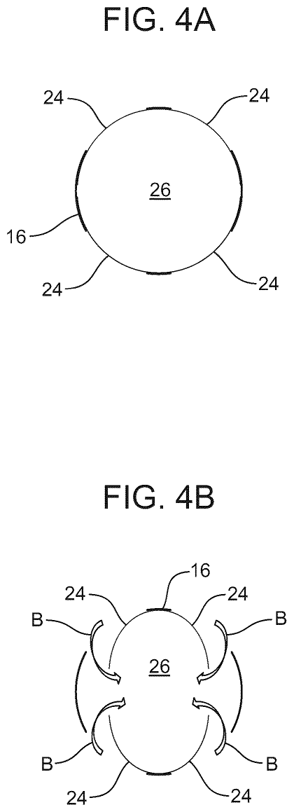

Reference is now made to FIGS. 2-10 which clearly illustrate the new and improved ventricular assist device 10 for ventricle support that employs a minimally invasive single cannulation technique for implantation. The device 10 includes a cannula 12 illustrated in detail in FIGS. 2, 3A and 3B. In one possible embodiment, the cannula 12 may be made to have an overall length of about 212.5 mm with an inner diameter of about 7 mm (21 Fr) and an outer diameter of about 7.7 mm (23 Fr). The wall thickness may be about 0.35 mm.

The cannula 12 may be made from any appropriate medical grade material including, for example, polyurethane by means of dip molding. Super elastic nickel-titanium memory alloy 14 (e.g. Nitinol, 0.0762 mm thickness and 0.508 mm width) may be molded into the cannula 12 during the polyurethane dipping process for reinforcement.

A one-way inlet valve 16 and a one-way outlet valve 18 are incorporated into the cannula 12 which may be of one piece construction to maintain integrity/reliability in use. As illustrated, the outlet valve 18 is provided at the first or distal end 20 of the cannula 12 while the inlet valve 16 is provided at an intermediate point of the cannula between the first or distal end and the second or proximal end 22 opposite the distal end.

As illustrated in detail in FIGS. 3A and 3B, the inlet valve 16 may comprise two bileaflet polyurethane membrane inlet valves that can be dip molded into the cannula wall. FIG. 3A shows the valve 16 and the leaflets 24 in the closed position sealing off the lumen 26 in the cannula 12. In contrast, FIG. 3B shows the valve 16 and the leaflets 24 in the open position allowing communication to the lumen in the cannula 12. Note action arrows A representing blood flow through the inlet valve 16 into the lumen 26.

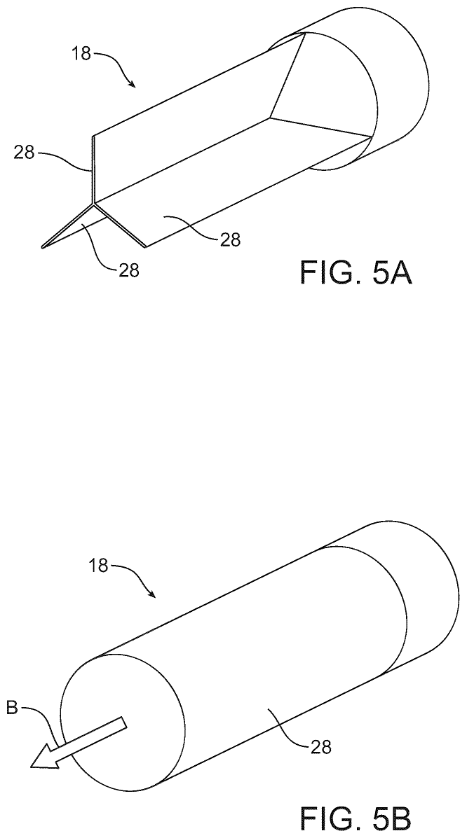

FIGS. 4A and 4B illustrate an alternative embodiment of inlet valve 16 comprising two “French door” bileaflet inlet valves. FIG. 4A shows the alternative embodiment of the valve 16 and the leaflets 24 in the closed position sealing off the lumen 26 in the cannula 12. In contrast, FIG. 4B shows the valve 16 and the leaflets 24 in the open position allowing communication to the lumen in the cannula 12. Note action arrows B representing blood flow through the inlet valve 16 into the lumen 26.

In either of the embodiments shown in FIGS. 3A, 3B or FIGS. 4A, 4B, the leaflets 24 open toward the lumen 26 during pump diastolic phase to allow for low resistant blood withdrawal from the heart ventricle through the inlet valve 16 into the lumen 26. In contrast, the leaflets 24 close during pump systolic phase to prevent blood back flow from the lumen 26 into the ventricle.

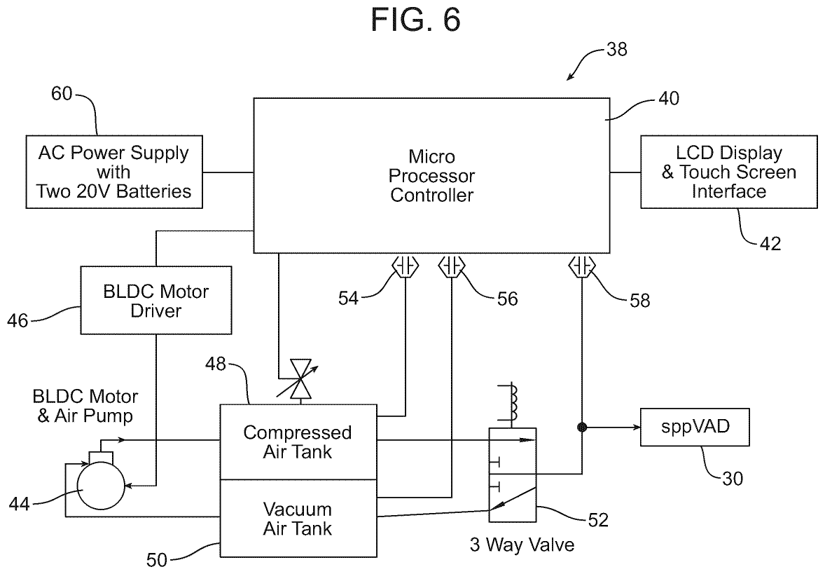

As illustrated in detail in FIGS. 5A and 5B, the outlet valve 18 may comprise a highly efficient duckbill valve that includes three leaflets 28 that operate in unison. FIG. 5A shows the outlet valve 18 and the leaflets 28 in the closed position sealing off the lumen 26 in the cannula 12. In contrast, FIG. 5B shows the outlet valve 18 and the leaflets 28 in the open position allowing communication to the lumen 26 of the cannula 12.

The outlet valve 18 regulates blood perfusion from the cannula into the aorta or pulmonary artery in a manner described in greater detail below. More specifically, during pump diastolic phase, the outlet valve 18 and leaflets 28 are closed to prevent blood flow back from the aorta or pulmonary artery into the cannula 12. In contrast, during systolic phase, the outlet valve 18 and the leaflets 28 are open to allow for perfusion of blood into the aorta or pulmonary artery.

While not illustrated, the outlet valve 18 of an alternative embodiment of the device 10 may also include two additional one-way outlet valves in the side wall of the cannula 12 adjacent duckbill valve at the distal end or tip of the device.

A pump 30 is connected to the cannula 12 at the second end 22 so as to be in communication with the lumen 26. The pump 30 may comprise a valveless, single port diaphragm displacement pump of a type known in the art. Such a pump 30 has a relatively inexpensive and simple structure that allows for reliable, efficient and trouble-free operation. As will be described in greater detail below, when the ventricular assist device 10 is properly implanted for use, the pump 30 is adapted to draw blood through the inlet valve 16 from the ventricle into the lumen 26 and also deliver blood from the lumen through the outlet valve 18 into the aorta or pulmonary artery thereby assisting and unloading the ventricle.

In one possible embodiment, the pump 30 has a 50 cc stroke volume. The rigid housing 32 of the pump 30 may be made in two halves by polycarbonate vacuum thermoforming and then assembled together with a flexible polyurethane membrane diaphragm (not shown) in the middle. The membrane diaphragm divides the housing 32 of the pump 30 into a blood chamber and a pneumatic chamber. A first port 34 in communication with the blood chamber is connected to the second or proximal end 22 of the cannula. A second port 36 in communication with the pneumatic chamber is connected to a pump drive 38 by means of pneumatic tubing 40.

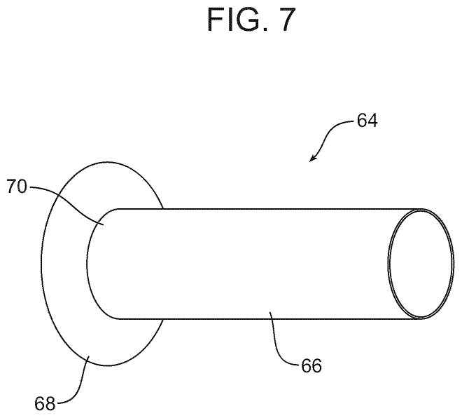

As best shown in FIG. 6, the pump drive 38 of the illustrated embodiment includes a microprocessor controller 40 in the form of a computing device that operates in accordance with dedicated hardware or appropriate software. In the illustrated embodiment, the controller 40 is connected to a liquid crystal display (LCD) and touch screen interface 42 to allow an operator to interface with the controller. The controller 40 controls operation of the air pump 44 through the motor driver 46. As shown, the motor 44 is connected to a compressed air tank 48 and a vacuum air tank 50 which, in turn, are connected to the pneumatic chamber of the pump 30 through a controller controlled three way valve 52. The controller 40 receives data from a positive pressure sensor 54, a vacuum sensor 56 and an output pressure sensor 58 to ensure proper operation at all times. Finally, the pump drive 38 is powered by the AC power supply 60 which includes two 20 volt batteries.

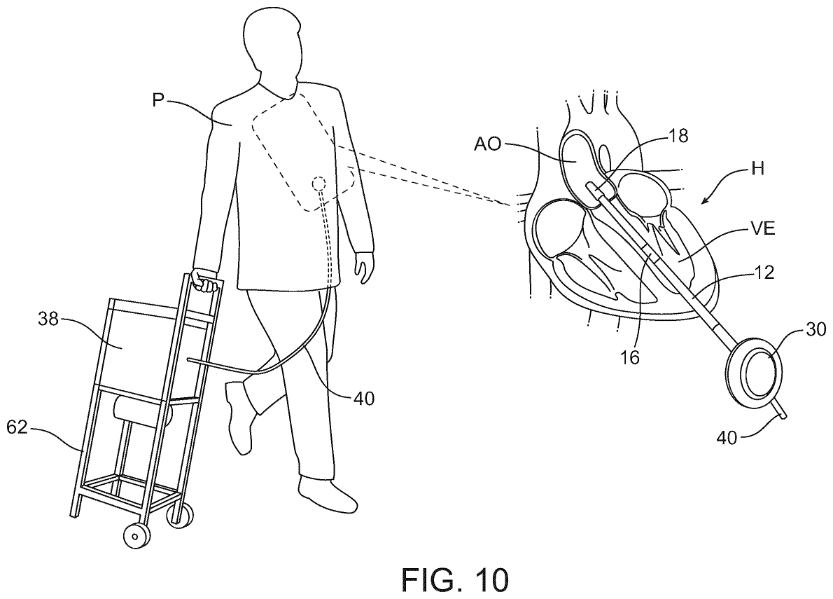

As shown in FIG. 10, the pump drive 38 may be carried on or mounted to a wheeled cart 62 to allow the cardiac shock patient to be ambulatory. Ambulation is an important factor in patient recovery. It can help reduce postoperative pain, improve fluid retention and prevent immune system issues. It can aid functional recovery by reducing or preventing muscle atrophy. It can prevent chest infections, strengthen muscles and joints and reduce the overall risk of surgery complications. All of these benefits tend to lead to a shorter hospital stay and reduced care costs.

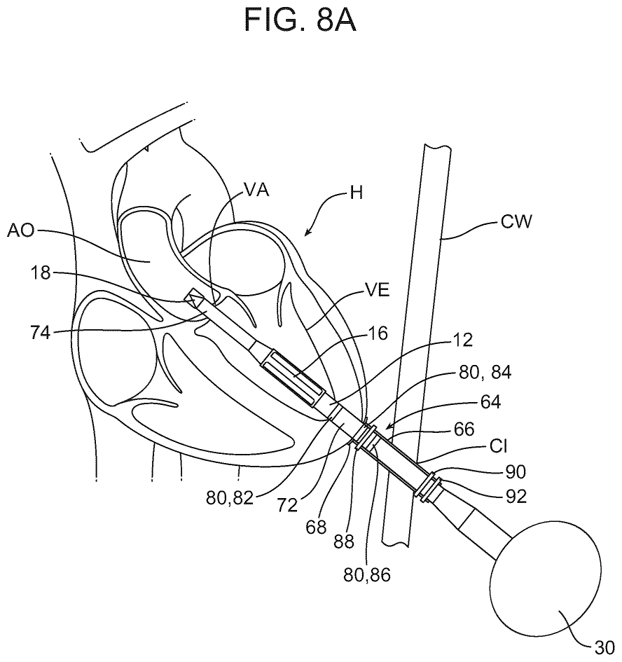

As illustrated in FIG. 7, the illustrated embodiment of the ventricular assist device 10 further includes an apical anchor 64. Anchor 64 includes a graft 66. As will be described in greater detail below, the graft 66 is adapted to receive and hold the cannula 12. The graft 66 may comprise a tube made from an appropriate medical grade synthetic material suited for this purpose.

An anchor retainer 68 is carried on a first end 70 of the graft 66. The anchor retainer 68 is adapted to secure the anchor 64 to the ventricle wall of the patient. The anchor retainer 68 may comprise a sewing mat made from appropriate medical grade material that is adapted to be sewn or stitched to the ventricle wall at, for example, the heart apex.

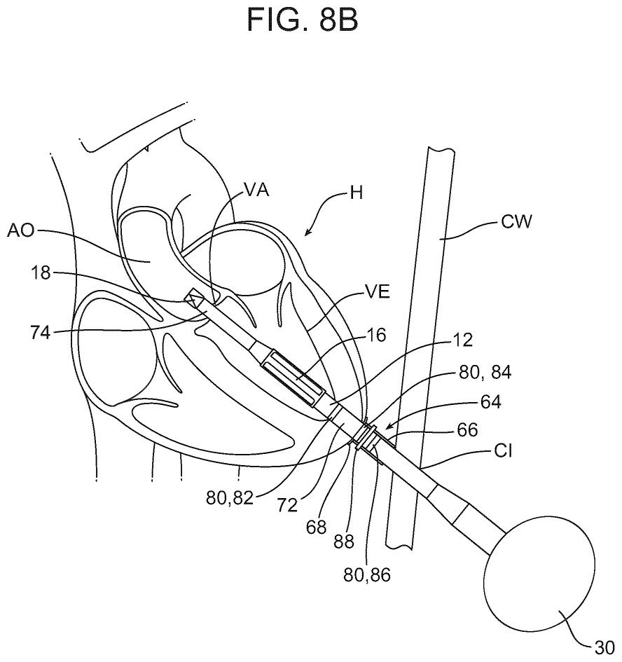

Reference is now made to FIG. 8A illustrating the proper implantation of the ventricular assist device 10 in the heart H of the patient P. When properly implanted, a first portion 72 of the cannula 12 is engaged with the wall of the left ventricle VE of the heart H into which the device 10 is implanted. At the same time, a second portion 74 of the cannula 12 crosses the valve VA between the ventricle VE and the aorta AO. As a result, the inlet valve 16 on the cannula 12 is positioned in the ventricle VE while the outlet valve 18 is positioned in the aorta AO. Thus, the valves 16, 18 are properly located to allow the pump 30 to (a) draw blood from the ventricle VE through the inlet valve 16 into the lumen 26 of the cannula 12 during the diastolic phase of operation and (b) deliver blood from the lumen in the cannula through the outlet valve to the aorta AO during the systolic phase of operation. As should be appreciated, the cannula 12 is held and retained in this proper operating position by the anchor 64 in a manner that will be described in detail below.

The ventricular assist device 10 is used in a method of providing mechanical circulatory support to the patient P. That method may be broadly described as including the steps of: (a) inserting the cannula 12 of a ventricular assist device 10 through a ventricle wall of the patient P, (b) positioning an inlet valve 16, carried on the cannula, in the ventricle VE and an outlet valve 18, carried on the cannula, in an aorta AO of the patient, (c) supporting the cannula with the anchor 64 and (d) establishing blood flow through the cannula with a pump 30 adapted to (a) draw blood from the ventricle through the inlet valve and (b) discharge blood through the outlet valve into the aorta.

More specifically, the step of inserting the cannula 12 includes preparing the device 10 for implantation. This includes connecting a flow probe and a pressure line between the cannula 12 and the pump 30 to measure pumping flow and pressure. The pump 30 is also pre-primed with heparinized saline. A catheter introducer, of a type known in the art, is then placed inside the lumen 26 of the cannula 12 with 10 cm of the catheter introducer extending out of the cannula tip at the first or distal end.

A mini-thoracotomy and a small pericardiotomy is then performed to expose the heart apex. A heparin bolus (150 U/kg, iv) is given to achieve an activated clotting time (ACT)>400 sec. Three pairs of mattress pledget stitches are then placed around the apex to sew the anchor retainer 68 of the anchor 64 on the heart apex.

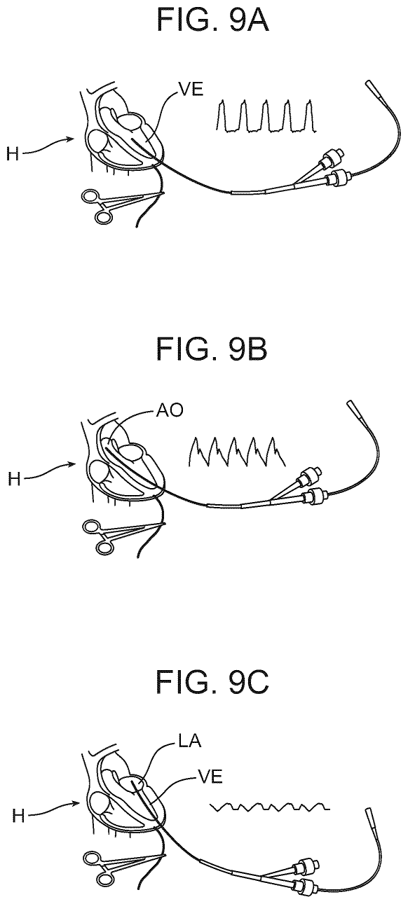

A 1 cm cut is then made inside the mattress stitch, and the catheter introducer is passed through the graft 66 and inserted into the ventricle VE through this cut. The catheter introducer is connected to a pressure transducer to measure tip pressure. The pressure waveform will guide catheter introducer tip advancement to the ascending aorta. See FIG. 9. FIG. 9A shows the waveform generated when the catheter introducer is in the left ventricle. FIG. 9B shows the waveform generated when the catheter introducer is in the ascending aorta. FIG. 9C shows the waveform generated when the catheter introducer is misplaced in the left atrium. The cannula 12 will slide along the catheter introducer through the apex cut, enter the left ventricle, cross the aortic valve, and will end with the outlet valve at the first end thereof in the ascending aorta AO when the waveform shown in FIG. 9B is presented.

As shown in FIG. 8A, a cannula retainer 80 is carried on the cannula 12. More particularly, in the illustrated embodiment, the cannula retainer takes the form of a first retainer ring 82, a second retainer ring 84 and a third retainer ring 86. When properly deployed, the graph 66 of the anchor 64 will be double tied (note ties 88) to the cannula 12 between the second and third retaining rings 84, 86 with the first retainer ring 82 inside the ventricle wall and the second retainer ring 84 outside the ventricle wall. The introducer is then removed and the cannula is connected to the pump 30. The pump 30 is connected to the pump drive 38 and pumping is then initiated with the pumping flow gradually increased until close to baseline cardiac output level.

The mini-thoracotomy is then closed. The graft 66 of the anchor 64, with the cannula 12 inside, is then brought out through chest incision CI. The graft 66 is then sewed with stitching to the skin of the chest wall at an intermediate point between the two ends of the graft. If desired, two additional ties 90, 92 may be used to further secure the cannula 12 to the graft 66 outside the chest wall CW for added security. All three of the retainer rings 82, 84 and 86 may be adjusted for positioning by sliding along the length of the cannula 12 in some embodiments. In other embodiments, one of more of the retaining rings 82, 84 and 86 are fixed to the cannula 12.

FIG. 8B illustrates an alternative embodiment where the graft 66 does not extend through the chest wall CW. The reference numbers used in FIG. 8B correspond to those same structures as identified above with reference to FIG. 8A.

Each of the following terms written in singular grammatical form: “a”, “an”, and “the”, as used herein, means “at least one”, or “one or more”. Use of the phrase “One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrase: “a tie”, as used herein, may also refer to, and encompass, a plurality of ties.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The phrase “consisting of”, as used herein, is closed-ended and excludes any element, step, or ingredient not specifically mentioned. The phrase “consisting essentially of”, as used herein, is a semi-closed term indicating that an item is limited to the components specified and those that do not materially affect the basic and novel characteristic(s) of what is specified.

Terms of approximation, such as the terms about, substantially, approximately, etc., as used herein, refers to ±10% of the stated numerical value.

In summary, numerous benefits and advantages are provided by the ventricular assist device 10 and the related method of providing mechanical circulatory support to a patient. The device may be implanted by a minimally invasive procedure through a small thoracotomy and a transapical to aorta cannulation. The device 10 provides total left ventricle support, achieving over 6 L/min mean pumping blood flow to ensure complete re-establishment of collapsed circulation. The device 10 also includes (a) a very small paracorporeal single port diaphragm displacement pump 30 connected to the heart through only one small transcutaneous, valved, single lumen cannula 12 and (b) a pump drive 38 carried on a wheeled cart 62 that facilitate very convenient ambulation that allows patient discharge from the ICU and even hospital for home temporary mechanical circulatory support.

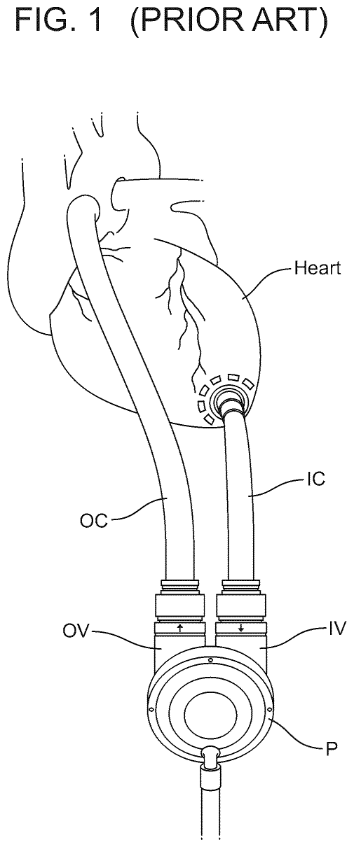

The single luman cannula 12 replaces both the inlet and outlet cannulas of the traditional diaphragm displacement pump shown in FIG. 1 to increase cannula usability rate from 50% to 100%. The integrated one-way inlet and outlet valves 16, 18 enable the use of a single lumen cannula 12 for both blood withdrawal and blood infusion for a simple/short circuit. In addition, the single lumen design maximizes the usable cannula cross-sectional area and dramatically shortens the circuit length, significantly reducing resistance to blood flow. Only one transapical cannulation allows fast, minimally invasive installation via a mini thoracotomy. Further, the cannula 12 is secured to the heart apex by the anchor 64 to resist or prevent dislodgement.

The valveless pump 30 replaces the two-valved and ported pump P of the prior art device shown in FIG. 1. The one valveless port 34 for both the withdrawal and infusion of blood eliminates the dead space found in the prior art pump device and dramatically decreases the size of the pump. The valveless design of the device 10 significantly simplifies pump geometry resulting in: (a) an increase in durability for longer term mechanical circulatory support and (b) almost no residual blood inside the pump 30 at the end of systole, significantly reducing thrombosis potential.

Finally, the new device 10 has easy ambulation for at-home mechanical circulatory support as a result of its single cannula design, cannula 12 secured in proper position at apex to prevent dislodgement and very small pump 30 in the paracorporeal position. This significantly reduces the hospital stay and the costs associated therewith.

Although the ventricular assist device 10, the anchor 64 and the method of providing mechanical circulatory support to a patient of this disclosure have been illustratively described and presented by way of specific exemplary embodiments, and examples thereof, it is evident that many alternatives, modifications, or/and variations, thereof, will be apparent to those skilled in the art. For example, the ventricular assist device 10 and anchor 64 may be used to assist the right ventricle by making a right thoracotomy through which the cannula is inserted through the right ventricle wall and then positioning the inlet valve 16 on the cannula 12 in the right ventricle and the outlet valve 18 on the cannula in the pulmonary artery. Accordingly, as should be appreciated, it is intended that all such alternatives, modifications, or/and variations, fall within the spirit of, and are encompassed by, the broad scope of the appended claims.

1. A ventricular assist device for a patient, comprising:
a cannula including a lumen;
an inlet valve on the cannula and in communication with the lumen;
an outlet valve on the cannula and in communication with the lumen;
a pump connected to the cannula and adapted to draw blood through the inlet valve from a ventricle of the patient into the lumen and deliver blood from the lumen through the outlet valve into an aorta or pulmonary artery of the patient; and
an anchor including (a) a graft adapted to receive and hold the cannula and (b) an anchor retainer carried on a first end of the graft and adapted to secure the anchor to a wall of the ventricle.

2. The ventricular assist device of claim 1, further including a cannula retainer carried on the cannula.

3. The ventricular assist device of claim 2, wherein the anchor further includes at least one tie fastening the graft to the cannula adjacent the cannula retainer.

4. The ventricular assist device of claim 2, wherein the cannula retainer includes a first retainer ring and a second retainer ring carried on the cannula wherein the first retainer ring is positioned inside the ventricle wall and the second retainer ring is positioned outside the ventricle wall.

5. The ventricular assist device of claim 4, wherein the anchor further includes at least one tie fastening the graft to the cannula between the second retainer ring and a third retainer ring.

6. The ventricular assist device of claim 5, further including stitching connecting the anchor retainer to the ventricle wall.

7. The ventricular device of claim 6, wherein the graft is adapted to extend from the anchor retainer connected to the ventricle wall through a chest wall of a patient into which the ventricular assist device is implanted.

8. The ventricular assist device of claim 7, further including stitching securing the graft to the chest wall at an intermediate point between the first end and a second opposite end of the graft.

9. The ventricular assist device of claim 1, wherein the anchor retainer is a sewing mat that is stitched to the ventricle wall.

10. The ventricular assist device of claim 1, wherein the graft is a tube made from a medical grade synthetic material.

11. The ventricular assist device of claim 10, wherein the cannula is received and held in the tube.

12. The ventricular assist device of claim 1, wherein the inlet valve is a one-way valve, the outlet valve is a one-way valve and the lumen is a single lumen.

13. The ventricular assist device of claim 12, wherein the pump is a valveless, single port diaphragm displacement pump.

14. The ventricular assist device of claim 13, further including a pump drive connected to the pump.

15. The ventricular assist device of claim 1, further including a first retainer ring, a second retainer ring and a third retainer ring carried on the cannula.

16. An anchor, comprising:
a graft adapted to receive and hold a heart cannula; and
an anchor retainer carried on a first end of the graft and adapted to secure the anchor to a ventricle wall of a patient.

17. The anchor of claim 16, further including at least one tie connecting the graft to the cannula.

18. The anchor of claim 17, further including stitching connecting the anchor retainer to the ventricle wall.

19. The anchor of claim 18, wherein the graft is adapted to extend from the anchor retainer connected to the ventricle wall through a chest wall of the patient.

20. The anchor of claim 19, further including stitching securing the graft to the chest wall at an intermediate point between the first end and a second opposite end of the graft.

21-36. (canceled)
